SEQUENCE LISTING (23 sequences)

SEQ ID NO: 1 - protein, 15 aa, Artificial Sequence
  Synthetic Peptide
KGGNGEPRGDTYRAY

SEQ ID NO: 2 - protein, 12 aa, Artificial Sequence
  Snythetic Peptide
NGEPRGDTYRAY

SEQ ID NO: 3 - protein, 7 aa, Artificial Sequence
  Synthetic Peptide
GRGDSPK

SEQ ID NO: 4 - protein, 12 aa, Artificial Sequence
  Snythetic Peptide
AVTGRGDSPASS

SEQ ID NO: 5 - protein, 12 aa, Artificial Sequence
  Snythetic Peptide
PQVTRGDVFTMP

SEQ ID NO: 6 - protein, 10 aa, Artificial Sequence
  Synthetic Peptide
RNIAEIIKDI

SEQ ID NO: 7 - protein, 15 aa, Artificial Sequence
  Synthetic Peptide
KYGRKRLQVQLSIRT

SEQ ID NO: 8 - protein, 11 aa, Artificial Sequence
  Synthetic Peptide
NGEPRGDTRAY

SEQ ID NO: 9 - protein, 12 aa, Artificial Sequence
  Synthetic Peptide
NGEPRGDTYRAY

SEQ ID NO: 10 - protein, 15 aa, Artificial Sequence
  Snythetic Peptide
KYGAASIKVAVSADR

SEQ ID NO: 11 - protein, 15 aa, Artificial Sequence
  Synthetic Peptide
KYGKAFDITYVRLKF

SEQ ID NO: 12 - protein, 16 aa, Artificial Sequence
  Synthetic Peptide
KYGSETTVKYIFRLHE

SEQ ID NO: 13 - protein, 16 aa, Artificial Sequence
  Synthetic Peptide
KYGTDIRVTLNRLNTF

SEQ ID NO: 14 - protein, 12 aa, Artificial Sequence
  Synthetic Peptide
TSIKIRGTYSER

SEQ ID NO: 15 - protein, 12 aa, Artificial Sequence
  Synthetic Peptide
TWYKIAFQRNRK

SEQ ID NO: 16 - protein, 20 aa, Artificial Sequence
  Synthetic Peptide
SINNNRWHSIYITRFGNMGS

SEQ ID NO: 17 - protein, 13 aa, Artificial Sequence
  Synthetic Peptide
KYGLALERKDHSG

SEQ ID NO: 18 - protein, 17 aa, Artificial Sequence
  Synthetic Peptide
GQKCIVQTTSWSQCSKS

SEQ ID NO: 19 - protein, 17 aa, Artificial Sequence
  Synthetic Peptide
KGGKDGEPRGDTYRATD

SEQ ID NO: 20 - protein, 13 aa, Artificial Sequence
  Synthetic Peptide
KGGLEPRGDTYRD

SEQ ID NO: 21 - protein, 17 aa, Artificial Sequence
  Synthetic Peptide
KGGCNGEPRGDTYRATC

SEQ ID NO: 22 - protein, 13 aa, Artificial Sequence
  Synthetic Peptide
KGGCEPRGDTYRC

SEQ ID NO: 23 - protein, 15 aa, Artificial Sequence
  Synthetic Peptide
KGGAVTGDGNSPASS

(19) United States
(12) Patent Application Publication
(10) Pub. No.: US 2011/0027888 A1
(43) Pub. Date: Feb. 3, 2011

(54) Coated Fibers for Culturing Cells

(76) Inventors: James P. Beltzer, Elmira, NY (US); Michelle Dawn Fabian, Horseheads, NY (US); Edward John Fewkes, Horseheads, NY (US); Kevin Robert McCarthy, Horseheads, NY (US); Florence Verrier, Corning, NY (US)

Correspondence Address: CORNING INCORPORATED, SP-TI-3-1, CORNING, NY, 14831, US

(21) Appl. No.: 12/837,763

(22) Filed: Jul. 16, 2010

Related U.S. Application Data
(60) Provisional application No. 61/229,339, filed on Jul. 29, 2009.

(51) Int. Cl. C12N 5/02; B29C 55/30; B05D 3/02

(52) U.S. Cl. 435/397; 264/129; 427/385.5; 435/289.1

(57) ABSTRACT

A coated fiber for cell culture includes a fiber core having an exterior surface and a polymeric coating suitable for culturing cells disposed on at least a portion of the exterior surface of the fiber core. A polypeptide may be conjugated to the polymeric coating. A method for forming the coated fiber includes coating a polymer layer to an exterior surface of a fiber core to produce the coated fiber. The coating may occur as the fiber is being drawn.

### CLAIMING BENEFIT OF PRIOR FILED U.S. APPLICATION

This application claims the benefit of U.S. Provisional Application Ser. No. 61/229,339, filed on Jul. 29, 2009. The content of this document and the entire disclosure of publications, patents, and patent documents mentioned herein are incorporated by reference.

### FIELD

The present disclosure relates to cell culture, and more particularly to coated fibers for use in cell culture and methods for manufacturing such fibers.

### BACKGROUND

Cell culture holds enormous potential for cell-based therapies, drug discovery and research. Scale up of anchorage dependent cell lines is typically achieved through the use of microcarriers which provide increased surface area for cell growth as compared to well plates, flasks, or roller bottles. Microcarriers are small spheres that are typically in the range of 100-500 microns in diameter. Microcarriers are typically coated with an animal derived coating such as Matrigel prior to use. Such microcarriers provide increased surface area of scaled-up cell culture. However, microcarriers do have difficulties associated with their use. Because of the low density required to keep them suspended in the culture medium, they can be difficult to separate from the medium when it is time to remove them from the assay. Also, in order to increase surface area, the size of the bead must be decreased which leads to excessive curvature, which may not be suitable for many anchorage dependent cells.

### BRIEF SUMMARY

Among other things, the present disclosure describes coated fibers that provide a three-dimensional surface for scaled-up cell culture. In various embodiments, the fibers are coated with a swellable (meth)acrylate layer that may be useful for large scale culture of hESCs. Processes for producing such coated fibers are also described herein.

In various embodiments, a coated fiber includes a fiber core having an exterior surface and a polymeric coating suitable for culturing cells disposed on at least a portion of the exterior surface of the fiber core. The coated fiber may further include a polypeptide conjugated to the coating.

In various embodiments, a method for producing a coated fiber for use in cell culture includes coating a polymer layer to an exterior surface of a fiber core to produce the coated fiber. The coating may be applied as the fiber core is being drawn.

The coated fibers have coatings that are conducing to cell culture. In various embodiments, the coatings without conjugated polypeptide do not support cell attachment, while the same coatings with conjugated polypeptide support cell attachment.

One or more of the various embodiments presented herein provide one or more advantages over prior articles and systems for culturing cells. For example, synthetic coated fibers described herein have been shown to support cell adhesion without the need of animal derived biocoating which limits the risk of pathogen contamination. This is especially relevant when cells are dedicated to cell-therapies. Further, large scale culture of cells is possible with coated fibers as described herein. Such coated fibers may also be advantageously used for culturing cells when animal derived products such as collagen, gelatin, fibronectin, etc. are undesired or prohibited. The methods described herein allow for the preparation of coated fibers having a wide range of properties such as stiffness, swellability, and surface chemistries. Further, in various embodiments, processes associated with the production of optical fibers may be employed to allow for low cost fabrication compared to other microcarriers available in the market. For example, it may be possible to produce many kilometers of coated fiber very short timeframe. Further such methods may provide for improved coating uniformity and coating thickness control as compared to the use of other coating processes (solution coating, dip coating etc). Using a fiber draw process, variables such as coating modulus, coating thickness, and overall fiber diameter (adjust surface area) can easily be changed in a low cost manner. Such coated fibers can provide for ease of handling when changing cell culture media (as compared to using small low density beads), and may allow for simplified harvesting of cells by running fibers through a stripper similar to that used to remove the coatings from an optical fiber. These and other advantages will be readily understood from the following detailed descriptions when read in conjunction with the accompanying drawings.

### BRIEF DESCRIPTION OF THE DRAWINGS

FIG. 1 is a schematic drawing of a radial cross-section of an embodiment of a coated fiber.

FIG. 2 is a schematic drawing of a radial cross-section of an embodiment of a coated fiber with a conjugated polypeptide.

FIG. 3 is a schematic drawing of a longitudinal cross-section of an embodiment of a coated fiber.

FIG. 4 is a schematic drawing illustrating representative components of a system that may be used to draw and coat fibers.

FIGS. 5A-B are images of crystal violet stained uncoated fiber (A) and coated fiber (B).

FIGS. 6A-B are fluorescence images of an uncoated fiber (A) and a coated fiber with conjugated polypeptide (B).

FIGS. 7A-B are micrographs an uncoated fiber (A) and a coated fiber with conjugated polypeptide (B) cultured with HT-1080 cells.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

Polypeptide sequences are referred to herein by their one letter amino acid codes and by their three letter amino acid codes. These codes may be used interchangeably.

As used herein, “monomer” means a compound capable of polymerizing with another monomer, (regardless of whether the “monomer” is of the same or different compound than the other monomer), which compound has a molecular weight of less that about 1000 Dalton. In many cases, monomers will have a molecular weight of less than about 400 Dalton.

As used herein “peptide” and “polypeptide” mean a sequence of amino acids that may be chemically synthesized or may be recombinantly derived, but that are not isolated as entire proteins from animal sources. For the purposes of this disclosure, peptides and polypeptides are not whole proteins. Peptides and polypeptides may include amino acid sequences that are fragments of proteins. For example peptides and polypeptides may include sequences known as cell adhesion sequences such as RGD. Polypeptides may be of any suitable length, such as between three and 30 amino acids in length. Polypeptides may be acetylated (e.g. Ac-LysGlyGly) or amidated (e.g. SerLysSer-NH₂) to protect them from being broken down by, for example, exopeptidases. It will be understood that these modifications are contemplated when a sequence is disclosed.

As used herein, “equilibrium water content” refers to water-absorbing characteristic of a polymeric material and is defined and measured by equilibrium water content (EWC) as shown by Formula 1:

EWC(%)=[(Wgel−Wdry)/(Wgel)]*100. Formula 1

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. Accordingly, a coated fiber comprising a fiber core and a coating includes a coated fiber consisting essentially of, or consisting of, a fiber core and a coating.

The present disclosure describes, inter alia, synthetic coated fibers for culturing cells. In various embodiments, the coated fibers are configured to support proliferation and maintenance of undifferentiated stem cells in chemically defined media.

### 1. Coated Fiber

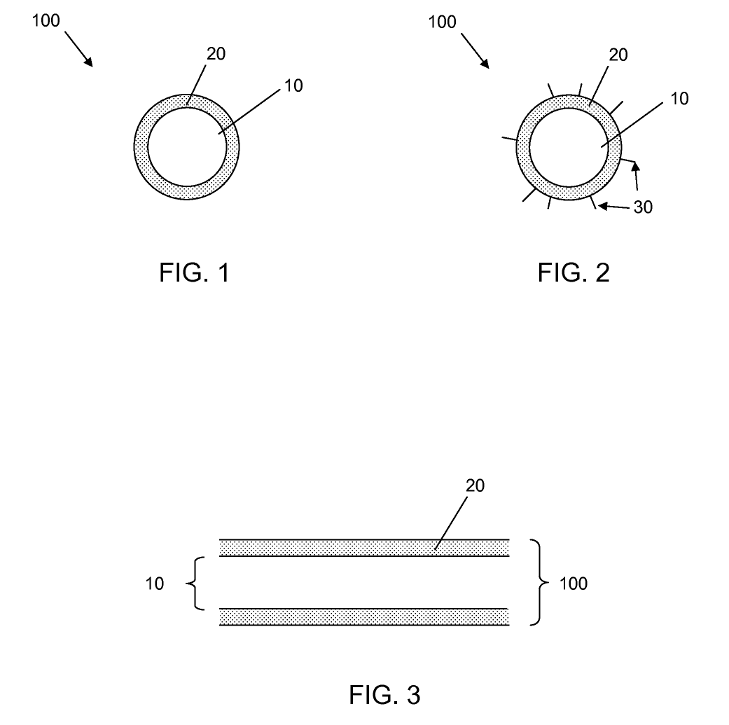

Referring to FIG. 1 and FIG. 2, schematic radial cross-sections of coated fibers 100 are shown. The depicted coated fiber 100 includes a solid fiber core 10 and a coating 20, and may include a conjugated polypeptide 30 (see FIG. 2). The coating 20 alone or coating 20 and polypeptide 30 together provide a surface to which cells can attach for the purposes of cell culture. In various embodiments, the coating layer 20 is deposited on or formed on a surface of an intermediate layer (not shown) that is associated with the core 10 via covalent or noncovalent interactions, either directly or via one or more additional intermediate layers (not shown). In such cases, the intermediate layer(s) is considered, for the purposes of this disclosure, to be a part of the fiber core 10.

FIG. 3 is a schematic longitudinal section of a coated fiber 100 showing the fiber core 10 and coating 20.

While the embodiments depicted in FIGS. 1-3 show the coating 20 disposed on or about the entire exterior surface of the fiber core 10, it will be understood that only a portion of the exterior surface of the fiber core 10 may be coated. Thus, the coated fiber 100 may include portions conducive to cell attachment or growth and portions not conducive to cell attachment and growth, as desired.

Coated fibers for purposes of culturing cells may be of any suitable dimension. For example, a coated fiber may have a diametric dimension of between about 50 microns and about 1000 microns, between about 100 microns and about 900 microns, or between about 125 microns and about 500 microns. A coated fiber may be sectioned or formed to any suitable length.

### 2. Fiber Core

Any suitable fiber core may be used. In various embodiments the fiber core is a drawn fiber, such as a drawn glass or polymeric fiber. Of course the fiber core may be formed of any other suitable material, such as a metallic material. Examples of polymeric materials that can be used to create drawn fibers include polyethylene, polypropylene, polycarbonate, nylon, polymethylmethacrylate (PMMA), polysulfone, cyclic olefin polymers, thermoplastic polyurethane, and polystyrene. In some embodiments, the fiber cores are drawn in a manner similar to that employed for drawing optical fibers, e.g. as described below in more detail. However, any suitable method may be employed to form the fiber core.

### 3. Coating

A fiber core may be coated with polymer from any suitable class of biocompatible polymers such as poly(meth)acrylates, polyamides, polyphosphazenes, polypropylfumarates, synthetic poly(amino acids), polyethers, polyacetals, polycyanoacrylates, polyacrylamides, polyurethanes, polycarbonates, polyanhydrides, poly(ortho esters), polyhydroxyacids, polyesters, ethylene-vinyl acetate polymers, cellulose acetates, polystyrenes, poly(vinyl chloride), poly(vinyl fluoride), poly(vinyl imidazole), poly(vinyl alcohol), chlorosulphonated polyolefins, and combinations thereof.

“Coating”, “layer”, “surface”, “material”, and the like are used interchangeably herein, in the context of a polymer disposed on a fiber core. Preferably, the coating is a synthetic polymer coating free from animal-derived components, as animal derived components occasionally may contain viruses or other infectious agents or may provide a high level of batch-to-batch variability. In various embodiments, the coating is a hydrophilic coating or a swellable (meth)acrylate coating, e.g., as described in U.S. patent application Ser. No. 12/362,924, filed on Jan. 30, 2009, entitled SYNTHETIC SURFACES FOR CULTURING CELLS IN CHEMICALLY DEFINED MEDIA, and having attorney docket no. SP08-018; and U.S. patent application Ser. No. 12/362,974, filed on Jan. 30, 2009, entitled SWELLABLE (METH)ACRYLATE SURFACES FOR CULTURING CELLS IN CHEMICALLY DEFINED MEDIA, and having attorney docket no. SP09-014, which applications are hereby incorporated herein by reference in their respective entireties to the extent that they do not conflict with the disclosure presented herein.

As used herein, “swellable (meth)acrylate” or “SA” means a polymer matrix made from at least one ethylenically unsaturated monomer (acrylate or methacrylate monomers) having at least some degree of cross linking, and also having water absorbing or water swelling characteristics. “SAP”, as used herein, means as SA conjugated to a polypeptide or protein. In embodiments, the term “swellable (meth)acrylate” represents a range of cross-linked acrylate or methacrylate materials which absorb water, swell in water, and do not dissolve in water.

In various embodiments, the SA coating comprises, consists essentially of, or consists of, reaction products of one or more hydrophilic (meth)acrylate monomer, one or more di- or higher-functional (meth)acrylate monomer (“cross-linking” (meth)acrylate monomer), and one or more carboxyl group-containing monomers. Any suitable hydrophilic (meth)acrylate monomer may be employed. Examples of suitable hydrophilic (meth)acrylate monomers include 2-hydroxyethyl methacrylate, di(ethylene glycol)ethyl ether methacrylate, ethylene glycol methyl ether methacrylate, and the like. In various embodiments, hydrophilic monomers other than (meth)acrylates may be used to form the SA coating. These other hydrophilic monomers may be included in addition to, or in place of, hydrophilic (meth)acrylate monomers. Such other hydrophilic monomers should be capable of undergoing polymerizing with (meth)acrylate monomers in the mixture used to form the swellable (meth)acrylate layer. Examples of other hydrophilic monomers that may be employed to form the SA coating include 1-vinyl-2-pyrrolidone, acrylamide, 3-sulfopropyldimethyl-3-methylacrylamideopropyl-ammonium, and the like. Regardless of whether a (meth)acrylate monomer or other monomer is employed, a hydrophilic monomer, in various embodiments, has a solubility in water of 1 gram or more of monomer in 100 grams of water. Any suitable di- or higher-functional (meth)acrylate monomer, such as tetra(ethylene glycol) dimethacrylate or tetra(ethylene glycol) diacrylate, may be employed as a cross-linking monomer. Any suitable (meth)acrylate monomer having a carboxyl functional group available for conjugating with a polypeptide after the monomer is incorporated into the SA coating by polymerization may be employed. The carboxyl functional group enables conjugation of a peptide or polypeptide using NHS/EDC chemistry. Examples of suitable carboxyl group-containing (meth)acrylate include 2-carboxyethyl acrylate and acrylic acid. In another embodiment, any suitable (meth)acrylate monomer having an epoxide group available for reaction with a polypeptide after the monomer is incorporated into the SA coating by polymerization may be employed. The epoxide group enables a direct nucleophilic addition reaction with an amine group on the polypeptide. An example of a suitable epoxide group containing (meth)acrylate monomer is glycidyl methacrylate.

In various embodiments, the SA layer is formed from monomers comprising (by percent volume): hydrophilic (meth)acrylate monomer (˜60-90), carboxyl group-containing (meth)acrylate monomer (˜10-40), and cross-linking (meth)acrylate monomer (˜1-10), respectively. It will be understood that the equilibrium water content (EWC) of the SA layer may be controlled by the monomers chosen to form the SA layer. For example, a higher degree of hydrophilicity and a higher percentage of the hydrophilic monomer should result in a more swellable SA layer with a higher EWC. However, this may be attenuated by increasing the percentage, or increasing the functionality, of the cross-linking monomer, which should reduce the ability of the SA layer to swell and reduce the EWC.

In various embodiments, the specific monomers employed to form the SA layer and their respective weight or volume percentages are selected such that the resulting SA layer has an EWC of between about 5% and about 70%. Due in part to the use of a carboxyl containing monomer in the SAs of various embodiments described herein, the EWC may be pH dependent. For example, the EWC of particular SAs may be higher in phosphate buffer (pH 7.4) than in distilled, deionized water (pH ˜5). In various embodiments, the EWC of an SA layer in distilled, deionized water is the EWC (in water) of SAs of the present invention may range between 5% and 70%, between 5% and 60%, between 5% and 50%, between 5 and 40%, between 5% and 35%, between 10% and 70%, between 10% and 50% between 10 and 40%, between 5% and 35%, between 10% and 35% or between 15% and 35% in water. In further embodiments, after the swellable (meth)acrylates have been conjugated with peptides (SAP), the EWC of embodiments of SAPs may be, for example, between 10-40% in water.

In cell culture, prepared surfaces are exposed to an aqueous environment for extended periods of time. Surfaces that absorb significant water, surfaces that are highly hydrogel-like, may tend to delaminate from a substrate when exposed to an aqueous environment. This may be especially true when these materials are exposed to an aqueous environment for extended periods of time, such as for 5 or more days of cell culture. Accordingly, it may be desirable for SA and SAP layers to have lower EWC measurements, so that they do not absorb as much water, to reduce the likelihood of delaminating. For example, SA surfaces having an EWC at or below 10%, at or below 15%, at or below 20%, at or below 25%, at or below 30%, at or below 35%, at or below 40%, at or below 45%, at or below 50%, at or below 55%, at or below 60% may be particularly suitable for supporting cells in culture, including human embryonic stem cells.

It will be understood that the conjugation of a polypeptide to an SA layer may affect the swellability and equilibrium water content (EWC) of the SA layer, generally increasing the EWC. The amount of polypeptide conjugated to SA layers tends to be variable and can change depending on the thickness of the SA layer. Accordingly, the EWC of a SA-polypeptide layers prepared in accordance with a standard protocol may be variable. For purposes of reproducibility, it may be desirable to measure the EWC of SA layers prior to conjugation with a polypeptide. With this noted, in some embodiments, after the SAs have been conjugated with polypeptides (SA-polypeptide), the EWC of embodiments of SA-polypeptide layers may be between about 10% and about 40% in water.

In various embodiments, the SA layer includes polymerized (meth)acrylate monomers formed from a mixture including hydroxyethyl methacrylate, 2-carboxyethylacrylate, and tetra(ethylene glycol) dimethacrylate. In numerous embodiments, the ratio (by volume) of hydroxyethyl methacrylate, 2-carboxyethylacrylate, and tetra(ethylene glycol) dimethacrylate used to form the SA layer is about 80/20/3 (v/v/v), respectively. In some embodiments, the SA is formulated using the following liquid aliquots of monomers (by volume): hydroxyethyl methacrylate (−20-90), 2-carboxyethylacrylate (−10-40), and tetra(ethylene glycol) dimethacrylate (−1-60), respectively. In numerous embodiments, the SA layer consists essentially of polymerized hydroxyethyl methacrylate, 2-carboxyethylacrylate, and tetra(ethylene glycol) dimethacrylate monomers. In various embodiments, the SA layer is substantially free of polypeptide crosslinkers.

Some representative swellable (meth)acrylate formulations that may be employed are illustrated in Table 1

TABLE 1
Swellable (meth)acrylate formulations

| | | Carboxyl group | |
|---|---|---|---|
| Formualtion | Hydrophilic Monomer | containing | Crosslinking |
| No. | (vol. %) | monomer (vol. %) | monomer (vol. %) |
| 1 | hydroxyethyl | 2-carboxyethyl | Tetra(ethylene |
| | methacrylate (80) | acrylate (20) | glycol) |
| | | | dimethacrylate (3) |
| 2 | hydroxyethyl | 2-carboxyethyl | Tetra(ethylene |
| | methacrylate (60) | acrylate (40) | glycol) |
| | | | dimethacrylate (3) |
| 3 | poly(ethylene | 2-carboxyethyl | |
| | glycol)(600) | acrylate (20) | |
| | dimethacrylate (80) | | |
| 4 | hydroxyethyl | 2-carboxyethyl | Tetra(ethylene |
| | methacrylate (90) | acrylate (10) | glycol) |
| | | | dimethacrylate (3) |
| 5 | hydroxyethyl | 2-carboxyethyl | Tetra(ethylene |
| | methacrylate (70) | acrylate (30) | glycol) |
| | | | dimethacrylate (3) |
| 6 | Hydroxypropyl | 2-carboxyethyl | Tetra(ethylene |
| | methacrylate(80) | acrylate (20) | glycol) |
| | | | dimethacrylate (3) |
| 7 | 2-Hydroxyethyl | 2-carboxyethyl | Tetra(ethylene |
| | acrylate(80) | acrylate (20) | glycol) |
| | | | dimethacrylate (3) |
| 9 | Di(ethylene glycol) ethyl | 2-carboxyethyl | Tetra(ethylene |
| | ether methacrylate(80) | acrylate (20) | glycol) |
| | | | dimethacrylate (3) |
| 11 | Ethylene glycol methyl | 2-carboxyethyl | Tetra(ethylene |
| | ether methacrylate(80) | acrylate (20) | glycol) |
| | | | dimethacrylate (3) |

A polymer coating layer may have any desirable thickness. In various embodiments, the average thickness of the coating layer is less than about 25 micrometers. For example, the average thickness may be less than about 20 micrometers, less than about 10 micrometers, and less than about 8 micrometers.

4. Coating of Fiber Base with Polymer

A polymer layer may be disposed on a surface of a fiber core via any known or future developed process. Preferably, the coating provides a uniform layer that does not delaminate during typical cell culture conditions. The coating layer may be associated with the fiber core via covalent or non-covalent interactions. Examples of non-covalent interactions that may associate the coating layer with the fiber core include chemical adsorption, hydrogen bonding, surface interpenetration, ionic bonding, van der Waals forces, hydrophobic interactions, dipole-dipole interactions, mechanical interlocking, and combinations thereof.

In numerous embodiments, a coating is deposited on a surface of a fiber core and polymerized in situ. Polymerization may be done in bulk phase. If it is desirable to reduce viscosity of the monomers, the temperature of the coating composition may be increased.

In addition to the monomers that form the coating layer, a composition forming the layer may include one or more additional compounds such as oligomers, surfactants, wetting agents, polymerization initiators, catalysts or activators.

When employed, suitable oligomers can be either monofunctional oligomers or polyfunctional oligomers. The oligomeric component can also be a combination of a monofunctional oligomer and a polyfunctional oligomer.

Di-functional oligomers preferably have a structure according to formula (I) below:

F₁—R₁-[Diisocyanate-R₂-Diisocyanate]ₘ-R₁—F₁ (I)

where
F₁ is independently a reactive functional group such as acrylate, methacrylate, acrylamide, N-vinyl amide, styrene, vinyl ether, vinyl ester, or other functional group known in the art;
R₁ includes, independently, —C₂₋₁₂O—, —(C₂₋₄O)ₙ—, —C₂₋₁₂O—(C₂₋₄ —O)ₙ—, —C₂₋₁₂O—(CO—C₂₋₅O)ₙ—, or —C₂₋₁₂O—(CO—C₂₋₅NH)ₙ— where n is a whole number from 1 to 30, or in embodiments 1 to 10;
R₂ is polyether, polyester, polycarbonate, polyamide, polyurethane, polyurea, or combinations thereof; and
m is a whole number from 1 to 10, or in additional embodiments 1 to 5.

In the structure of formula I, the diisocyanate group is the reaction product formed following bonding of a diisocyanate to R₂ or R₁. The term “independently” is used herein to indicate that each F₁ may differ from another F₁ and the same is true for each R₁.

Other polyfunctional oligomers preferably have a structure according to formula (II), formula (III), or formula (IV) as set forth below:

multiisocyanate-(R₂—R₁—F₂)ₓ (II);

polyol-[(diisocyanate-R₂-diisocyanate)ₘ-R₁—F₂]ₓ (III); or

multiisocyanate-(R₁—F₂)ₓ (IV)

where
F₂ independently represents from 1 to 3 functional groups such as acrylate, methacrylate, acrylamide, N-vinyl amide, styrene, vinyl ether, vinyl ester, or other functional groups known in the art;
R₁ can include —C₂₋₁₂O—, —(C₂₋₄—O)ₙ—, —C₂₋₁₂O—(C₂₋₄—O)ₙ—, —C₂₋₁₂O—(CO—C₂₋₅O)ₙ—, or —C₂₋₁₂O—(CO—C₂₋₅NH)ₙ-where n is a whole number from 1 to 10, or in embodiments 1 to 5;
R₂ can be polyether, polyester, polycarbonate, polyamide, polyurethane, polyurea or combinations thereof;
x is a whole number from 1 to 10, or in embodiments 2 to 5; and
m is a whole number from 1 to 10, or in embodiments 1 to 5.

In the structure of formula II, the multiisocyanate group is the reaction product formed following bonding of a multiisocyanate to R₂. Similarly, the diisocyanate group in the structure of formula III is the reaction product formed following bonding of a diisocyanate to R₂ or R₁.

Urethane oligomers are conventionally provided by reacting an aliphatic diisocyanate with a dihydric polyether or polyester, most typically a polyoxyalkylene glycol such as a polyethylene glycol. Such oligomers typically have between about four to about ten urethane groups and may be of high molecular weight, e.g., 2000-8000. However, lower molecular weight oligomers, having molecular weights in the 500-2000 range, may also be used. When it is desirable to employ moisture-resistant oligomers, they may be synthesized in an analogous manner, except that the polar polyether or polyester glycols are avoided in favor of predominantly saturated and predominantly nonpolar aliphatic diols. These diols include, for example, alkane or alkylene diols of from about 2-250 carbon atoms and, preferably, are substantially free of ether or ester groups. As is well known, polyurea components may be incorporated in oligomers prepared by these methods, simply by substituting diamines or polyamines for diols or polyols in the course of synthesis.

Any suitable polymerization initiator may be employed. One of skill in the art will readily be able to select a suitable initiator, e.g. a photoinitiator suitable for use with the monomers. Suitable photoinitiators include, without limitation, 2,4,6-Trimethylbenzoyldiphenylphosphine oxide (e.g. Lucirin TPO), 1-hydroxycyclohexylphenyl ketone (e.g.; Irgacure 184 available from Ciba Specialty Chemical (Tarrytown, N.Y.)), (2,6-diethoxybenzoyl)-2,4,4-trimethylpentyl phosphine oxide (e.g. in commercial blends Irgacure 1800, 1850, and 1700, Ciba Specialty Chemical), 2,2-dimethoxyl-2-phenyl acetophenone (e.g., Irgacure, 651, Ciba Specialty Chemical), bis(2,4,6-trimethylbenzoyl)phenyl phosphine oxide (e.g., Irgacure 819, Ciba Specialty Chemical), (2,4,6-triiethylbenzoyl)diphenyl phosphine oxide (e.g., in commercial blend Darocur 4265, Ciba Specialty Chemical), 2-hydroxy-2-methyl-1-phenylpropane-1-one (e.g., in commercial blend Darocur 4265, Ciba Specialty Chemical) and combinations thereof. Other photoinitiators are continually being developed and used in coating compositions on glass fibers. Any suitable photoinitiator can be introduced into compositions of the present invention.

A photosensitizer may also be included in a suitable initiator system. Representative photosensitizers have carbonyl groups or tertiary amino groups or mixtures thereof. Photosensitizers having a carbonyl groups include benzophenone, acetophenone, benzil, benzaldehyde, o-chlorobenzaldehyde, xanthone, thioxanthone, 9,10-anthraquinone, and other aromatic ketones. Photosensitizers having tertiary amines include methyldiethanolamine, ethyldiethanolamine, triethanolamine, phenylmethylethanolamine, and dimethylaminoethylbenzoate. Commercially available photosensitizers include QUANTICURE ITX, QUANTICURE QTX, QUANTICURE PTX, QUANTICURE EPD from Biddle Sawyer Corp.

In general, the amount of photosensitizer or initiator in a composition may vary from about 0.01 to 10% by weight.

When polymerized, the monomers are polymerized via an appropriate initiation mechanism. Many of such mechanisms are known in the art. For example, temperature may be increased to activate a thermal initiator, photoinitiators may be activated by exposure to appropriate wavelength of light, or the like. Polymerization may be carried out under inert gas protection, such as nitrogen protection, to prevent oxygen inhibition.

The cured coating layer may be washed with solvent one or more times to remove impurities such as unreacted monomers or low molecular weight polymer species. In various embodiments, the layer is washed with ethanol or an ethanol/water solution, e.g. 50% ethanol, 70% ethanol, greater than 90% ethanol, greater than 95% ethanol or greater than about 99% ethanol.

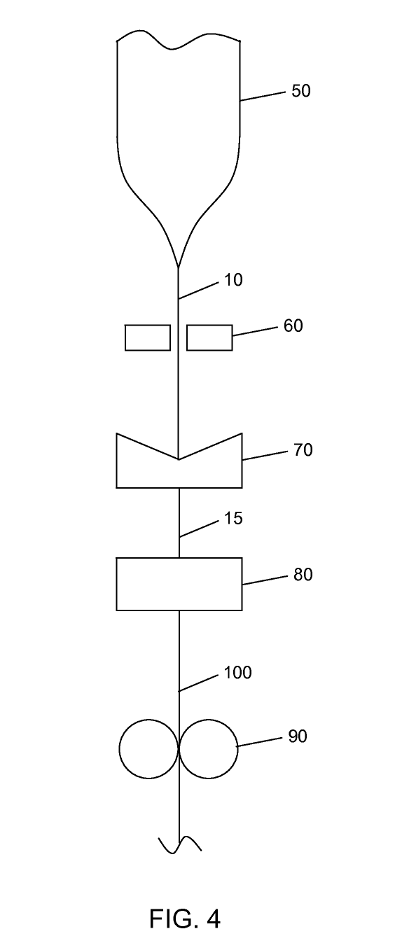

Referring now to FIG. 4, the polymer layer, in the depicted embodiment is coated on the fiber core 10 as the fiber is being drawn. Any suitable system for drawing fibers may be employed, and it will be understood that components other than or different from those depicted in FIG. 4 may be employed. The system depicted in FIG. 4 is shown for purposes of illustration. Briefly, the fiber drawing system of FIG. 4 includes a perform 50, and a pair of tractors 90 for drawing the fiber 10 from the perform 50, which may be melted or molten. The system may include an optical micrometer 60, whose output may be coupled to a control system that regulates the speed of the tractors 90 to control fiber 10 diameter. The drawn fiber core 10 may then pass through a coater which houses a coating die to control the thickness of the coating layer 70, which may house a coating composition, and exits as a coated fiber core 15, which then may pass through a suitable curing apparatus 80. The curing apparatus 80 employed will depend on the nature of the monomers and polymerization initiators used. For example, if the initiators are photoinitiators, the curing apparatus 80 may include a UV lamp; if the initiators are thermal initiators, the curing apparatus 80 may include a heater; etc. After curing the coated fiber 100 may be sectioned to the appropriate length for use in cell culture.

### 5. Polypeptides

Any suitable polypeptide may be conjugated to a coated fiber. In various embodiments, polypeptides or proteins are synthesized or obtained through recombinant techniques, making them synthetic, non-animal-derived materials. Preferably, polypeptide includes an amino acid capable of conjugating to the coating; e.g. via the free carboxyl group formed from a monomer used to form the coating. By way of example, any native or biomimetic amino acid having functionality that enables nucleophilic addition; e.g. via amide bond formation, may be included in polypeptide for purposes of conjugating to the coating. Lysine, homolysine, ornithine, diaminoproprionic acid, and diaminobutanoic acid are examples of amino acids having suitable properties for conjugation to a carboxyl group of the fiber. In addition, the N-terminal alpha amine of a polypeptide may be used to conjugate to the carboxyl group, if the N-terminal amine is not capped. In various embodiments, the amino acid of polypeptide that conjugates with the coating is at the carboxy terminal position or the amino terminal position of the polypeptide.

In numerous embodiments, the polypeptide, or a portion thereof, has cell adhesive activity; i.e., when the polypeptide is conjugated to the coated fiber, the polypeptide allows a cell to adhere to the surface of the peptide-containing coated fiber. By way of example, the polypeptide may include an amino sequence, or a cell adhesive portion thereof, recognized by proteins from the integrin family or leading to an interaction with cellular molecules able to sustain cell adhesion. For example, the polypeptide may include an amino acid sequence derived from collagen, keratin, gelatin, fibronectin, vitronectin, laminin, bone sialoprotein (BSP), or the like, or portions thereof. In various embodiments, polypeptide includes an amino acid sequence of ArgGlyAsp (RGD).

Coated fibers as described herein provide a synthetic surface to which any suitable adhesion polypeptide or combinations of polypeptides may be conjugated, providing an alternative to biological substrates or serum that have unknown components. In current cell culture practice, it is known that some cell types require the presence of a biological polypeptide or combination of peptides on the culture surface for the cells to adhere to the surface and be sustainably cultured. For example, HepG2/C3A hepatocyte cells can attach to plastic culture ware in the presence of serum. It is also known that serum can provide polypeptides that can adhere to plastic culture ware to provide a surface to which certain cells can attach. However, biologically-derived substrates and serum contain unknown components. For cells where the particular component or combination of components (peptides) of serum or biologically-derived substrates that cause cell attachment are known, those known polypeptides can be synthesized and applied to a fiber as described herein to allow the cells to be cultured on a synthetic surface having no or very few components of unknown origin or composition.

For any of the polypeptides discussed herein, it will be understood that a conservative amino acid may be substituted for a specifically identified or known amino acid. A “conservative amino acid”, as used herein, refers to an amino acid that is functionally similar to a second amino acid. Such amino acids may be substituted for each other in a polypeptide with a minimal disturbance to the structure or function of the polypeptide according to well known techniques. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing Methionine (M), Cysteine (C); Basic: Arginine (R), Lysine (K), Histidine (H); Acidic: Aspartic acid (D), Glutamic acid (E), Asparagine (N), Glutamine (Q).

A linker or spacer, such as a repeating poly(ethylene glycol) linker or any other suitable linker, may be used to increase distance from polypeptide to surface of the coated fiber. The linker may be of any suitable length. For example, if the linker is a repeating poly(ethylene glycol) linker, the linker may contain between 2 and 10 repeating ethylene glycol units. In some embodiments, the linker is a repeating poly(ethylene glycol) linker having about 4 repeating ethylene glycol units. All, some, or none of the polypeptides may be conjugated to a coated fiber via linkers. Other potential linkers that may be employed include polypeptide linkers such as poly(glycine) or poly(β-alanine).

A polypeptide may be conjugated to the coated fiber at any density, preferably at a density suitable to support culture of undifferentiated stem cells or other cell types. Polypeptides may be conjugated to a fiber at a density of between about 1 pmol per mm² and about 50 pmol per mm² of surface of the fiber. For example, the polypeptide may be present at a density of greater than 5 pmol/mm², greater than 6 pmol/mm², greater than 7 pmol/mm², greater than 8 pmol/mm², greater than 9 pmol/mm², greater than 10 pmol/mm², greater than 12 pmol/mm², greater than 15 pmol/mm², or greater than 20 pmol/mm² of the surface of the coated fiber. It will be understood that the amount of polypeptide present can vary depending on the composition of the coating of the fiber, the size of the fiber and the nature of the polypeptide itself.

A polypeptide may be conjugated to the coated fiber via any suitable technique. A polypeptide may be conjugated to a coated fiber via an amino terminal amino acid, a carboxy terminal amino acid, or an internal amino acid. One suitable technique involves 1-ethyl-3-[3-dimethylaminopropyl]carbodiimide hydrochloride (EDC)/N-hydroxysuccinimide (NHS) chemistry, as generally known in the art. EDC and NHS or N-hydroxysulfosuccinimide (sulfo-NHS) can react with carboxyl groups of the swellable (meth)acrylate layer to produce amine reactive NHS esters. EDC reacts with a carboxyl group of the coating layer to produce an amine-reactive O-acylisourea intermediate that is susceptible to hydrolysis. The addition of NHS or sulfo-NHS stabilizes the amine-reactive O-acylisourea intermediate by converting it to an amine reactive NHS or sulfo-NHS ester, allowing for a two step procedure. Following activation of the coating, the polypeptide may then be added and the terminal amine of the polypeptide can react with the amine reactive ester to form a stable amide bond, thus conjugating the polypeptide to the coating. When EDC/NHS chemistry is employed to conjugate a polypeptide to the coating, the N-terminal amino acid is preferably an amine containing amino acid such as lysine, ornithine, diaminobutyric acid, or diaminoproprionic acid. Of course, any acceptable nucleophile may be employed, such as hydroxylamines, hydrazines, hydroxyls, and the like.

EDC/NHS chemistry results in a zero length crosslinking of polypeptide to fiber. Linkers or spacers, such as poly(ethylene glycol) linkers (e.g., available from Quanta BioDesign, Ltd.) with a terminal amine may be added to the N-terminal amino acid of polypeptide. When adding a linker to the N-terminal amino acid, the linker is preferably a N-PG-amido-PEG_{X}-acid where PG is a protecting group such as the Fmoc group, the BOC group, the CBZ group or any other group amenable to peptide synthesis and X is 2, 4, 6, 8, 12, 24 or any other discrete PEG which may be available.

In various embodiments, a 1 μM-2500 μM polypeptide fluid composition, such as a solution, suspension, or the like, is contacted with an activated coated fiber to conjugate the polypeptide. For example the polypeptide concentration may be between about 100 μM and about 2000 μM, between about 500 μM and about 1500 μM, or about 1000 μM. It will be understood that the volume of the polypeptide composition and the concentration may be varied to achieve a desired density of polypeptide conjugated to the fiber.

The polypeptide may be cyclized or include a cyclic portion. Any suitable method for forming cyclic polypeptide may be employed. For example, an amide linkage may be created by cyclizing the free amino functionality on an appropriate amino-acid side chain and a free carboxyl group of an appropriate amino acid side chain. Also, a di-sulfide linkage may be created between free sulfydryl groups of side chains appropriate amino acids in the peptide sequence. Any suitable technique may be employed to form cyclic polypeptides (or portions thereof). By way of example, methods described in, e.g., WO1989005150 may be employed to form cyclic polypeptides. Head-to-tail cyclic polypeptides, where the polypeptides have an amide bond between the carboxy terminus and the amino terminus may be employed. An alternative to the disulfide bond would be a diselenide bond using two selenocysteines or mixed selenide/sulfide bond, e.g., as described in Koide et al, 1993, Chem. Pharm. Bull. 41(3):502-6; Koide et al., 1993, Chem. Pharm. Bull. 41(9):1596-1600; or Besse and Moroder, 1997, Journal of Peptide Science, vol. 3, 442-453.

Polypeptides may be synthesized as known in the art (or alternatively produced through molecular biological techniques) or obtained from a commercial vendor, such as American Peptide Company, CEM Corporation, or GenScript Corporation. Linkers may be synthesized as known in the art or obtained from a commercial vendor, such as discrete polyethylene glycol (dPEG) linkers available from Quanta BioDesign, Ltd.

An example of a polypolypeptide that may be conjugated to a fiber is a polypeptide that includes KGGNGEPRGDTYRAY (SEQ ID NO:1), which is an RGD-containing sequence from bone sialoprotein with an additional “KGG” sequence added to the N-terminus. The lysine (K) serves as a suitable nucleophile for chemical conjugation, and the two glycine amino acids (GG) serve as spacers. Cystine (C), or another suitable amino acid, may alternatively be used for chemical conjugation, depending on the conjugation method employed. Of course, a conjugation or spacer sequence (KGG or CGG, for example) may be present or absent. Additional examples of suitable polypeptides for conjugation with fiber (with or without conjugation or spacer sequences) are polypeptides that include NGEPRGDTYRAY, (SEQ ID NO:2), GRGDSPK (SEQ ID NO:3) (short fibronectin) AVTGRGDSPASS (SEQ ID NO:4) (long FN), PQVTRGDVFTMP (SEQ ID NO:5) (vitronectin), RNIAEIIKDI (SEQ ID NO:6) (lamininβ1), KYGRKRLQVQLSIRT (SEQ ID NO:7) (mLMα1 res 2719-2730), NGEPRGDTRAY (SEQ ID NO:8) (BSP—Y), NGEPRGDTYRAY (SEQ ID NO:9) (BSP), KYGAASIKVAVSADR (SEQ ID NO:10) (mLMα1 res 2122-2132), KYGKAFDITYVRLKF (SEQ ID NO:11) (mLMγ1 res 139-150), KYGSETTVKYIFRLHE (SEQ ID NO:12) (mLMγ1 res 615-627), KYGTDIRVTLNRLNTF (SEQ ID NO:13) (mLMγ1 res 245-257), TSIKIRGTYSER (SEQ ID NO:14) (mLMγ1 res 650-261), TWYKIAFQRNRK (SEQ ID NO:15) (mLMα1 res 2370-2381), SINNNRWHSIYITRFGNMGS (SEQ ID NO:16) (mLMα1 res 2179-2198), KYGLALERKDHSG (SEQ ID NO:17) (tsp1 RES 87-96), or GQKClVQTTSWSQCSKS (SEQ ID NO:18) (Cyr61 res 224-240).

In some embodiments, the peptide comprises KGGK⁴DGEPRGDTYRATD¹⁷ (SEQ ID NO:19), where Lys⁴ and Asp¹⁷ together form an amide bond to cyclize a portion of the polypeptide; KGGL⁴EPRGDTYRD¹³ (SEQ ID NO:20), where Lys⁴ and Asp¹³ together form an amide bond to cyclize a portion of the polypeptide; KGGC⁴NGEPRGDTYRATC¹⁷ (SEQ ID NO:21), where Cys⁴ and Cys¹⁷ together form a disulfide bond to cyclize a portion of the polypeptide; KGGC⁴EPRGDTYRC¹³ (SEQ ID NO:22), where Cys⁴ and Cys¹³ together form a disulfide bond to cyclize a portion of the polypeptide, or KGGAVTGDGNSPASS (SEQ ID NO:23).

In embodiments, the polypeptide may be acetylated or amidated or both. While these examples are provided, those of skill in the art will recognize that any peptide or polypeptide sequence may be conjugated to a fiber as described herein.

### 6. Incubating Cells in Culture Media Having Coated Fiber

Coated fibers as described herein may be used in any suitable cell culture system. The coated fibers and cell culture media may be placed in a suitable cell culture article. Suitable cell culture articles include bioreactors, such as the WAVE BIOREACTOR® (Invitrogen), single and multi-well plates, such as 6, 12, 96, 384, and 1536 well plates, jars, petri dishes, flasks, multi-layered flasks, beakers, plates, roller bottles, tubes, bags, membranes, cups, spinner bottles, perfusion chambers, bioreactors, CellSTACK® culture chambers (Corning Incorporated) and fermenters.

A cell culture article housing culture media containing a coated fiber described above may be seeded with cells. The coated fiber employed may be selected based on the type of cell being cultured. The cells may be of any cell type. For example, the cells may be connective tissue cells, epithelial cells, endothelial cells, hepatocytes, skeletal or smooth muscle cells, heart muscle cells, intestinal cells, kidney cells, or cells from other organs, stem cells, islet cells, blood vessel cells, lymphocytes, cancer cells, primary cells, cell lines, or the like. The cells may be mammalian cells, preferably human cells, but may also be non-mammalian cells such as bacterial, yeast, or plant cells.

In numerous embodiments, the cells are stem cells which, as generally understood in the art, refer to cells that have the ability to continuously divide (self-renewal) and that are capable of differentiating into a diverse range of specialized cells. In some embodiments, the stem cells are multipotent, totipotent, or pluripotent stem cells that may be isolated from an organ or tissue of a subject. Such cells are capable of giving rise to a fully differentiated or mature cell types. A stem cell may be a bone marrow-derived stem cell, autologous or otherwise, a neuronal stem cell, or an embryonic stem cell. A stem cell may be nestin positive. A stem cell may be a hematopoietic stem cell. A stem cell may be a multi-lineage cell derived from epithelial and adipose tissues, umbilical cord blood, liver, brain or other organ. In various embodiments, the stem cells are pluripotent stem cells, such as pluripotent embryonic stem cells isolated from a mammal Suitable mammals may include rodents such as mice or rats, primates including human and non-human primates. In various embodiments, the coated fiber with conjugated polypeptide supports undifferentiated culture of embryonic stem cells for 5 or more passages, 7 or more passages, or 10 or more passages. Typically stems cells are passaged to a new surface after they reach about 75% confluency. The time for cells to reach 75% confluency is dependent on media, seeding density and other factors as know to those in the art.

Because human embryonic stem cells (hESC) have the ability to grown continually in culture in an undifferentiated state, the hESC for use with the coated fiber as described herein may be obtained from an established cell line. Examples of human embryonic stem cell lines that have been established include, but are not limited to, H1, H7, H9, H13 or H14 (available from WiCell established by the University of Wisconsin) (Thompson (1998) Science 282:1145); hESBGN-01, hESBGN-02, hESBGN-03 (BresaGen, Inc., Athens, Ga.); HES-1, HES-2, HES-3, HES-4, HES-5, HES-6 (from ES Cell International, Inc., Singapore); HSF-1, HSF-6 (from University of California at San Francisco); I 3, I 3.2, I 3.3, I 4, I 6, I 6.2, J 3, J 3.2 (derived at the Technion-Israel Institute of Technology, Haifa, Israel); UCSF-1 and UCSF-2 (Genbacev et al., Fertil. Steril. 83(5):1517-29, 2005); lines HUES 1-17 (Cowan et al., NEJM 350(13):1353-56, 2004); and line ACT-14 (Klimanskaya et al., Lancet, 365(9471):1636-41, 2005). Embryonic stem cells may also be obtained directly from primary embryonic tissue. Typically this is done using frozen in vitro fertilized eggs at the blastocyst stage, which would otherwise be discarded.

Other sources of pluripotent stem cells include induced primate pluripotent stem (iPS) cells. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst as well as surface antigen expression, gene expression and telomerase activity resembling blastocyst derived embryonic stem cells. The iPS cells typically have the ability to differentiate into at least one cell type from each of the primary germ layers: ectoderm, endoderm and mesoderm. The iPS cells, like hESC, also form teratomas when injected into immuno-deficient mice, e.g., SCID mice. (Takahashi et al., (2007) Cell 131(5):861; Yu et al., (2007) Science 318:5858).

To maintain stem cells in an undifferentiated state it may be desirable to minimize non-specific interaction or attachment of the cells with the surface of the coated fiber, while obtaining selective attachment to the polypeptide(s) attached to the surface. The ability of stem cells to attach to the surface of a coated fiber without conjugated polypeptide may be tested prior to conjugating polypeptide to determine whether the fiber provides for little to no non-specific interaction or attachment of stem cells. Once a suitable coated fiber has been selected, cells may be seeded in culture medium containing the fiber.

Prior to seeding cells, the cells, regardless or cell type, may be harvested and suspended in a suitable medium, such as a growth medium in which the cells are to be cultured once seeded. For example, the cells may be suspended in and cultured in a serum-containing medium, a conditioned medium, or a chemically-defined medium. As used herein, “chemically-defined medium” means cell culture media that contains no components of unknown composition. Chemically defined cell culture media may, in various embodiments, contain no proteins, hydrosylates, or peptides of unknown composition. In some embodiments, chemically defined media contains polypeptides or proteins of known composition, such as recombinant growth hormones. Because all components of chemically-defined media have a known chemical structure, variability in culture conditions and thus variability in cell response can be reduced, increasing reproducibility. In addition, the possibility of contamination is reduced. Further, the ability to scale up is made easier due, at least in part, to the factors discussed above. Chemically defined cell culture media are commercially available from Invitrogen (Invitrogen Corporation, 1600 Faraday Avenue, PO Box 6482, Carlsbad, Calif. 92008) as STEM PRO, a fully serum- and feeder-free (SFM) specially formulated from the growth and expansion of embryonic stem cells, Xvivo (Lonza), and Stem Cell Technologies, Inc. as mTeSR™1 maintenance media for human embryonic stem cells.

One or more growth or other factors may be added to the medium in which cells are incubated with the fiber conjugated to polypeptide. The factors may facilitate cellular proliferation, adhesion, self-renewal, differentiation, or the like. Examples of factors that may be added to or included in the medium include muscle morphogenic factor (MMP), vascular endothelium growth factor (VEGF), interleukins, nerve growth factor (NGF), erythropoietin, platelet derived growth factor (PDGF), epidermal growth factor (EGF), activin A (ACT) such as activin A, hematopoietic growth factors, retinoic acid (RA), interferons, fibroblastic growth factors, such as basic fibroblast growth factor (bFGF), bone morphogenetic protein (BMP), peptide growth factors, heparin binding growth factor (HBGF), hepatocyte growth factor, tumor necrosis factors, insulin-like growth factors (IGF) I and II, transforming growth factors, such as transforming growth factor-β1 (TGFβ1), and colony stimulating factors.

The cells may be seeded at any suitable concentration. Typically, the cells are seeded at about 10,000 cells/cm² of fiber to about 500,000 cells/cm². For example, cells may be seeded at about 50,000 cells/cm² of substrate to about 150,000 cells/cm². However, higher and lower concentrations may readily be used. The incubation time and conditions, such as temperature, CO₂ and O₂ levels, growth medium, and the like, will depend on the nature of the cells being cultured and can be readily modified. The amount of time that the cells are cultured with the fiber may vary depending on the cell response desired.

The cultured cells may be used for any suitable purpose, including (i) obtaining sufficient amounts of undifferentiated stem cells cultured on a synthetic surface in a chemically defined medium for use in investigational studies or for developing therapeutic uses, (ii) for investigational studies of the cells in culture, (iii) for developing therapeutic uses, (iv) for therapeutic purposes, (v) for studying gene expression, e.g. by creating cDNA libraries, (vi) for studying drug and toxicity screening, and (vii) the like.

One suitable way to determine whether cells are undifferentiated is to determine the presence of the OCT4 marker. In various embodiments, the undifferentiated stems cells cultured on microcarriers as described herein for 5, 7, or 10 or more passages retain the ability to be differentiated.

In the following, non-limiting examples are presented, which describe various non-limiting embodiments of the coated fibers and methods discussed above.

### EXAMPLES

### Example 1

### Coating of Optical Fiber

A variety of coatings suitable for cell culture were applied to the exterior surfaces of optical fibers. Briefly, the fibers with an outer diameter of 245 micrometers were coated with a compositions having components are indicated in Table 2, where Irgacure 819 is Bis(2,4,6-trimethylbenzoyl)-phenylphosphineoxide, and Irgacure 184 is 1-Hydroxycyclohexyl phenylketone.

TABLE 2
Coating Compositions

| | Weight Percent | | |
|---|---|---|---|
| Component | Example 1 | Example 2 | Example 3 |
| 2-Hydroxyethyl | 77 | 60 | 20 |
| methacrylate | | | |
| 2-Carboxyethyl | 20 | 20 | 20 |
| acrylate | | | |
| Triethyleneglycol | 3 | 20 | 60 |
| diacrylate | | | |
| Irgacure 819 | 1.5 pph | 1.5 pph | 1.5 pph |
| Irgacure 184 | 1.5 pph | 1.5 pph | 1.5 pph |

To produce the coating compositions in Table 2, the appropriate amount of each monomer and initiator was weighed into a jacketed beaker and heated to 70° C. followed by mixing until the photoinitiators were completely dissolved.

The coatings were applied to an optical fiber using an optical fiber draw. Using compositions prepared as described in Table 2, the coatings were applied to drawn glass fibers with an outer diameter of 245 micrometers. The fibers were introduced into a coating chamber containing the coating compositions from Table 2. As the coated fiber was removed from the chamber the thickness of the coating layer was adjusted so that the cured coating thickness would be about 5 microns. The coating thickness was adjusted by passing the coated fiber through a die at a draw speed of 9 m/minute. Upon exiting the coating die, the fibers were exposed to UV radiation (using a Fusion D bulb, Fusion UV Systems, Inc.) on the draw to polymerize the coating.

### Example 2

### Crystal Violet Staining to Verify Fiber was Coated

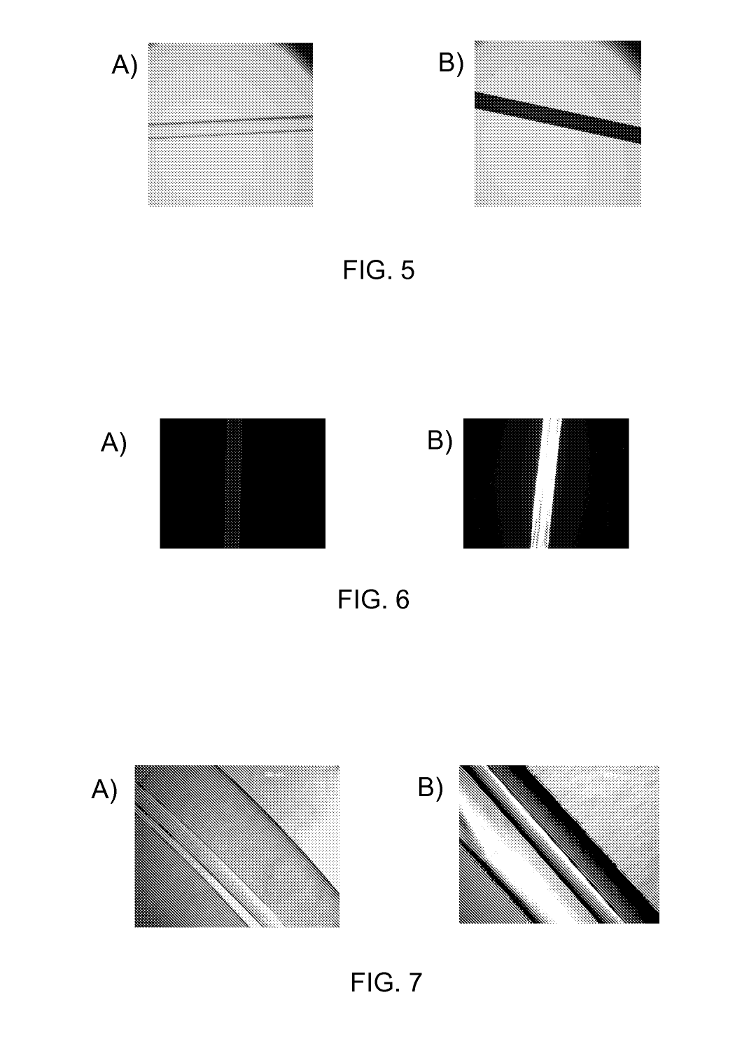

Crystal violet staining was used to verify that the fibers of EXAMPLE 1 were coated. Briefly, a small sample of coated fiber was placed in a solution of 2 mL centrifuge tube. 500 μL of a 1:5 dilution of crystal violet blue in water was added to the centrifuge tube. After 5 minutes, the sample was aspiration washed with DI water or until top solution was clear and colorless. Staining of the fiber was assessed using a light microscope. A fiber with no coating was also exposed to the crystal violet stain as a negative control. Representative images are shown in FIG. 5, where an uncoated fiber is shown in FIG. 5A, and a coated fiber is shown in FIG. 5B. The presence of the coating is confirmed by the crystal violet staining.

### Example 3

### Conjugation of Polypeptide to Coating

A vitrotronectin polypeptide (LysGlyGlyProGlnValThrArgGlyAspValPheThrMetPro (SEQ ID NO:5)) was conjugated to the surface of the coated fibers produced according to EXAMPLE 1. Briefly, 50 mg of coated fiber (250 micron outer diameter) was transferred to a 2 mL centrifuge tube. 94 mg of EDC (12 equiv, 191.70 g/mol, 492 μmol) and 14 mg NHS (3 equiv, 115 g/mol, 123 μmol) was dissolved in 1.5 mL of DMF and added to the fiber and allowed to mix on an orbital shaker for 60 min. The solution was aspirated, rinsed once with DMF, aspirated and then 1 mL of vitronectin peptide solution (10 mM in borate buffer, pH 9.2, 0.25% Rhodamine peptide spiked) was added and allowed mix for 60 min. The peptide solution was removed by aspiration and the fibers were treated with 1.5 mL of 1M ethanolamine pH 8 for 10 min followed by washing with PBS (1.5 mL×5), 1% SDS (1×1.5 mL×1.5 min), and DI Water and ethanol (1.5 mL×5) and dried under a gentle stream of nitrogen. For comparison, a fiber with no hydrogel coating was treated in the same manner to show specific binding of the peptide to the hydrogel coating.

Representative fluorescence micrographs (wavelength of 590 nm was used to excite rhodamine) are shown in FIG. 6, where FIG. 6A shows the fiber with no coating and FIG. 6B shows the coated fiber. The fluorescence in seen in FIG. 6B, confirms the conjugation of the polypeptide to the coating.

### Example 4

### Use of Coated Fiber in Cell Culture

HT1080 human fibrocarcoma cells were cultured on polypeptide-conjugated coated fibers produced according to EXAMPLE 3. Briefly, cells were trypsinized and allowed to recover in Iscove's Modified Dulbecco's Medium (IMDM) with 10% Fetal Bovine Serum (FBS) for 30 minutes at 37° C., 5% CO₂. After recovery, the cells were washed and resuspended in 0.1% Bovine Serum Albumin (BSA) in IMDM. Approximately 10 mg of peptide derivatized fiber was transferred to a 2 mL centrifuge tube and blocked with 2 mL of 1% BSA in D-PBS for 1 hr at room temperature. The fibers were then aspiration washed with 2 mL of D-PBS and incubated with 2 mL of 0.1% BSA in IMDM prior to cell seeding. 2 mL of re-suspended cells were placed in several wells of a 24 well Corning Ultra low attachment microplate. Approximately 10 mg of peptide coated fiber was added to each cell-seeded well, and the fiber/cell suspension was incubated for 1 hr at 37° C., 5% CO₂. The media was removed and the fibers were aspiration washed in the wells with D-PBS (2×2 mL). Cellular attachment and spreading was assessed using Ziess Axiovert 200M inverted microscope.

Representative images are shown in FIG. 7, where the image in FIG. 7A is of a negative control, uncoated optical fiber incubated with cells as described above, and the image is FIG. 7B is of a polypeptide-conjugated coated fiber. The polypeptide-conjugated coated fiber shows homogenous attachment along the fiber.

Thus, embodiments of SYNTHETIC MICROCARRIER FIBERS FOR CULTURING CELLS are disclosed. One skilled in the art will appreciate that the arrays, compositions, kits and methods described herein can be practiced with embodiments other than those disclosed. The disclosed embodiments are presented for purposes of illustration and not limitation.

What is claimed is:

1. A coated fiber for cell culture, comprising:
a fiber core having an exterior surface; and
a polymeric coating suitable for culturing cells disposed on at least a portion of the exterior surface of the fiber core.

2. A coated fiber according to claim 1, wherein the polymeric coating comprises a swellable (meth)acrylate layer formed from a composition comprising a carboxyl group-containing (meth)acrylate monomer, a cross-linking (meth)acrylate monomer, and a hydrophilic monomer capable of polymerizing with the carboxyl group-containing (meth)acrylate monomer and the cross-linking (meth)acrylate monomer

3. A coated fiber according to claim 1, further comprising a polypeptide conjugated to the coating.

4. A coated fiber according to claim 2, further comprising a polypeptide conjugated to the coating.

5. A coated fiber according to claim 3, wherein the polypeptide comprises an amino acid sequence of ArgGlyAsp.

6. A coated fiber according to claim 4, wherein the polypeptide comprises an amino acid sequence of ArgGlyAsp.

7. A method for forming a coated fiber according to claim 1, for use in cell culture, comprising:
coating a polymer layer to an exterior surface of a fiber core to produce the coated fiber.

8. A method according to claim 7, further comprising drawing a perform to form the fiber core.

9. A method according to claim 8, wherein coating the polymer layer to the exterior surface of the fiber core comprises coating the fiber core with the polymer layer while of fiber core is being drawn.

10. A method according to any of claim 7, wherein coating the polymer layer to the exterior surface of the fiber core comprises disposing monomers on the exterior surface of the fiber core and polymerizing the monomers on the fiber core to produce the polymer layer.

11. A method according to claim 7, wherein the polymer layer is formed from monomers comprising (i) a carboxyl group-containing (meth)acrylate monomer, (ii) a cross-linking (meth)acrylate monomer, and (iii) a hydrophilic monomer capable of polymerizing with the carboxyl group-containing (meth)acrylate monomer and the cross-linking (meth)acrylate monomer.

12. A method according to any of claim 7, further comprising a conjugating a polypeptide to the polymer layer.

13. A method according to claim 12, wherein the polypeptide comprises an amino acid sequence of ArgGlyAsp.

14. A method for culturing cells, comprising:
contacting the cells with a cell culture medium and a coated fiber, according to claim 1 the culturing the cells on the coated fiber in the medium.

15. A method according to claim 14, wherein the polymeric coating comprises a swellable (meth)acrylate layer formed from a composition comprising a carboxyl group-containing (meth)acrylate monomer, a cross-linking (meth)acrylate monomer, and a hydrophilic monomer capable of polymerizing with the carboxyl group-containing (meth)acrylate monomer and the cross-linking (meth)acrylate monomer.

16. A method according to claim 14, wherein the coated fiber comprises a polypeptide conjugated to the coating.

17. A method according to claim 15, where the coated fiber compresses a polypeptide conjugated to the coating.

18. A method according to claim 16, wherein the polypeptide comprises an amino acid sequence of ArgGlyAsp.

19. A method according to claim any of claim 14, wherein the cells comprise stem cells.

20. A method according to any of claim 14, wherein the cell culture medium is a chemically-defined medium.
